(19) United States
(12) Patent Application Publication
(10) Pub. No.: US 2018/0092815 A1
(43) Pub. Date: Apr. 5, 2018

(54) POLYMERIZABLE QUANTUM DOT NANOPARTICLES AND THEIR USE AS THERAPEUTIC, ABLATION AND TATTOOING AGENTS

(71) Applicant: Nanoco Technologies Ltd., Manchester (GB)

(72) Inventors: Imad Naasani, Manchester (GB); Mark Saunders, Manchester (GB)

(21) Appl. No.: 15/718,239

(22) Filed: Sep. 28, 2017

Related U.S. Application Data
(60) Provisional application No. 62/403,877, filed on Oct. 4, 2016.

(51) Int. Cl. A61K 8/19; A61K 8/02; A61Q 1/02; A61K 41/00; A61K 8/81

(52) U.S. Cl. CPC A61K 8/19; A61K 8/0245; B82Y 5/00; A61K 41/008; A61K 8/8152; A61Q 1/025

(57) ABSTRACT

The present disclosure relates to quantum dot nanoparticles conjugated to ligands, and in particular quantum dot nanoparticles wherein each nanoparticle is conjugated to a polymerizable ligand. The present disclosure also relates to methods of making such conjugated quantum dot nanoparticles, and the use of such conjugated quantum dot nanoparticles as therapeutic agents, ablation agents and tattooing agents.

### FIELD OF THE INVENTION

Embodiments disclosed herein relate to quantum dot nanoparticles conjugated to ligands, and in particular quantum dot nanoparticles wherein each nanoparticle is conjugated to a polymerizable ligand. Embodiments also include methods of making such conjugated quantum dot nanoparticles, and the use of such conjugated quantum dot nanoparticles as therapeutic agents, ablation agents and tattooing agents.

### BACKGROUND OF THE INVENTION

Tissue ablation is one of the therapeutic methods used to treat cancer. To date, it has been based on using laser, heat, microwave, radiofrequency and chemical (such as ethanol) agents as ablation tools. See e.g., Gillams, Cancer Imaging, 5, 103-109, 2005. However, such methods tend to lead to a lack of specificity and a high tendency to damage healthy surrounding tissues.

There has been substantial interest in the preparation and characterization of particles with dimensions, for example in the range 2-50 nm, often referred to as quantum dots or nanocrystals. Quantum dots (QDs) are fluorescent nanoparticles with unique optical properties including broad-range excitation, size-tunable emission, narrow emission bandwidth, enhanced brightness (due to high extinction coefficient), photo-stability, multiplexing capabilities, and simultaneous multiple emissions using a single source of excitation. Unlike normal fluorescent dyes, the unique properties of QDs enable several potential medical applications including unmet diagnostics, clinical imaging, targeted drug delivery, and photodynamic therapy.

Most solid tumors show specific uptake and retention for nanoparticles. This is attributed to the Enhanced Permeability and Retention effect (EPR). Nanoparticles on their own can offer tumor specific accumulation. Additional specificity can be achieved by equipping the nanoparticles with ligands that specifically bind to tumors. Nanoparticles on their own cannot induce tumor death or ablation unless they are functionalized for that purpose.

### SUMMARY OF THE INVENTION

There is a need for new ablation agents. This disclosure is based on using quantum dot nanoparticles as ablation agents. When water soluble quantum dots nanoparticles are equipped with polymerizable ligands, the excitation of the dots may cause dot-dot crosslinking that leads to intra-tissue aggregation and tissue necrosis or death.

Embodiments disclosed include quantum dot nanoparticles that may be used as agents for the visualization and treatment/ablation of cancer (e.g., pancreatic cancer, lung cancer, bladder cancer). Additional embodiments include quantum dot nanoparticles that may be used as tattooing agents.

Embodiments disclosed include quantum dot nanoparticles, wherein each nanoparticle is bonded (e.g., covalently bonded or physically bonded (by ion pairing or van der Waals interactions) to a polymerizable ligand, by, e.g., aliphatic chains, π-π stacking, π interactions, an amide, ester, thioester, or thiol anchoring group directly on an inorganic surface of the quantum dot nanoparticle, or on an organic corona layer that is used to render the nanoparticles water soluble and biocompatible. The water soluble quantum dot nanoparticle in certain embodiments includes a core of one semiconductor material and at least one shell of a different semiconductor material in some embodiments while in other embodiments the water soluble quantum dot nanoparticle includes an alloyed semiconductor material having a bandgap value that increases outwardly by compositionally graded alloying. Such embodiments are useful, for example, for the visualization and treatment/ablation of cancer, both in vitro and in vivo (e.g., in real time).

In one embodiment, each quantum dot nanoparticle is conjugated to a polymerizable ligand that may be polymerized once triggered by a chemical and/or physical action (e.g., excitation by a light source).

In one embodiment, each quantum dot nanoparticle described herein is covalently linked to a polymerizable ligand via an amide bond.

In one embodiment, each quantum dot nanoparticle comprises: a core semiconductor material, and an outer layer, wherein the outer layer comprises a corona of organic coating (a functionalization organic coating) to render the particles water soluble and bio compatible, and a polymerizable ligand. In one embodiment, each quantum dot nanoparticle comprises one or more shells of semiconductor material, the outer shell comprising an outer layer, wherein the outer layer comprises a corona of organic coating (a functionalization organic coating) to render the particles water soluble and bio compatible, and a polymerizable ligand.

In one embodiment, each quantum dot nanoparticle comprises: an alloyed quantum dot and a polymerizable ligand.

In one embodiment, each quantum dot nanoparticle comprises: a doped quantum dot and a polymerizable ligand.

Suitable polymerizable ligands include, but are not limited to, acrylates, methacrylates, diacetylene, cyanoacrylates, azide/alkyne pairs (click chemistry) and any combination thereof.

In one embodiment, the polymerizable ligand is a methacrylate (e.g., 2-aminoethyl methacrylate) or a salt thereof, such as a hydrochloride salt.

Suitable acryl based polymerizable ligands include, for example, methacryloyl-L-lysine, 4-methacryloxy-2-hydroxybenzophenone, and salts thereof, and any combination thereof.

In one embodiment, the polymerizable ligand comprises acrylate and methacrylate ligands. For example, quantum dot nanoparticles comprising methacrylate ligands may be polymerized and crosslinked using excitation light to induce exciton formation that can in turn initiate acrylate polymerization.

In additional embodiments, light active monomers (such as, e.g., methacryloyl-L-lysine, 4-methacryloxy-2-hydroxybenzophenone) may be used alone or in combination with one or more standard monomers to enhance the quantum dot nanoparticle's polymerization.

In one embodiment, the polymerizable ligand is a cyanoacrylate.

In one embodiment, the polymerizable ligand is glycidyl cinnamate, or a derivative thereof.

In another embodiment, the polymerizable ligand is a diacetylene, e.g., tricosa-10,12-diynoic acid.

In one embodiment, each quantum dot nanoparticle is conjugated to a polymerizable ligand based on the color of the fluorescence wavelength of the quantum dot (such as, e.g., green, yellow or red). It should be understood that each quantum dot nanoparticle described herein may be individually tuned to emit at a specific wavelength across the whole visible spectrum, and beyond, and can be tuned to the polymerizable ligand selected.

In one embodiment of any of the quantum dot nanoparticles described herein, the nanoparticle comprises a II-VI material, a III-V material, or I-III-IV material, or any alloy or doped derivative thereof.

In one embodiment, any of the quantum dot nanoparticles described herein are associated with an emission spectrum ranging from about 350 nm to about 1000 nm and further from about 450 nm to about 800 nm.

In an additional embodiment, any of the quantum dot nanoparticles described herein may further comprise a cellular uptake enhancer, (cell-penetrating peptides (CPPs like TAT, RGD, or poly arginine), a tissue penetration enhancer, (e.g., saponins, cationic lipids, Streptolysin O (SLO)), or any combination thereof. Examples of cellular uptake enhancers include, for example, trans-activating transcriptional activators (TAT), Arg-Gly-Asp (RGD) tri-peptides, or poly arginine peptides.

In another embodiment, a method of inducing cell death is provided.

In another embodiment, a method of inducing cell death and imaging affected tissues is provided.

In another embodiment, a method of visualizing and treating tumors (both malignant and benign) is provided. In additional embodiments, the tumor is soft or solid.

In another embodiment, a method of ablating unwanted tissue (including, for example, varicose veins, telangiectasia, spider nevus (spider veins)) is provided.

In another embodiment, a method of cosmetic tattooing is provided.

In one embodiment, any of the methods described herein comprises i) contacting a quantum dot nanoparticle conjugates (e.g., a plurality or a panel of quantum dot nanoparticle conjugates) according to any of the embodiments described herein with a cell, tumor or unwanted tissue, and (ii) polymerizing the ligand (e.g., triggering polymerization by a chemical and/or physical action). In an additional aspect of the embodiment, the ligand is polymerized by excitation of the quantum dot nanoparticles with an energy source (e.g., a light source, such as a UV or visible light source).

In one embodiment of any of the methods described herein, the quantum dots are excited using a multi-photon (e.g., a two-photon excitation). In such an embodiment, the combined energy of two or more light beams is used to excite a particular quantum dot nanoparticle.

In one embodiment, any of the methods described herein are performed in bodily fluids (e.g., blood, pancreatic juice, plasma, fine needle aspirate) and/or tissues samples in vivo. In one embodiment, any of the methods described herein are performed in bodily fluids and/or tissues samples taken and examined in vitro.

In one embodiment, the ligand-nanoparticle conjugates are introduced to living tissue. In another embodiment, the ligand-nanoparticle conjugates are introduced to a mammal for real-time ablation of cancer.

In another aspect, the present invention provides the use of ligand-nanoparticle conjugates according to any of the embodiments described herein for inducing cell death upon polymerization.

In another aspect, the present invention provides the use of ligand-nanoparticle conjugates according to any of the embodiments described herein for inducing cell death upon polymerization and imaging affected tissues.

In another aspect, the present invention provides the use of ligand-nanoparticle conjugates according to any of the embodiments described herein for the visualization and treatment of malignant and benign tumors.

In another aspect, the present invention provides the use of ligand-nanoparticle conjugates according to any of the embodiments described herein for the visualization and treatment of soft and solid tumors.

In another aspect, the present invention provides the use of ligand-nanoparticle conjugates according to any of the embodiments described herein for cosmetic tattooing.

In another aspect, the present invention provides the use of ligand-nanoparticle conjugates according to any of the embodiments described herein for ablating unwanted tissues, varicose veins, telangiectasia, and spider nevus (spider veins).

### BRIEF DESCRIPTION OF THE DRAWINGS

FIGS. 1A and 1B depict examples showing the polymerization of quantum dot nanoparticles conjugated to methacryloyl-L-lysine and 4-methacryloxy-2-hydroxybenzophenone before and after exposure to UV light.

FIG. 2 depicts an exemplary mechanism for the uptake and polymerization of quantum dot nanoparticles in cancer cells.

FIG. 3 depicts an example showing the polymerization of quantum dot nanoparticles conjugated to a polymerizable ligand and taken up by cells before and after exposure to UV light.

FIG. 4 depicts an example showing the polymerization of quantum dot nanoparticles conjugated to a polymerizable ligand and taken up by cells before and after exposure to UV light.

### DETAILED DESCRIPTION OF THE INVENTION

Disclosed herein are Quantum Dots (QDs) conjugated with cancer specific binding ligands that have the ability to be detected upon stimulation of the QD under conditions resulting in photon emission by the QD. Also disclosed herein are certain embodiments that provide QDs that feature high safety and biocompatibility profiles and are conjugated with polymerizable ligands. In certain embodiments, the QD is engineered as a conjugate of biocompatible, non-toxic, fluorescent QDs.

ABBREVIATIONS: To facilitate the understanding of this invention, and for the avoidance of doubt in construing the claims herein, a number of terms are defined below. Terms defined herein have meanings as commonly understood by a person of ordinary skill in the areas relevant to the present invention. The terminology used to describe specific embodiments of the invention does not delimit the invention, except as outlined in the claims.

DCC dicyclohexylcarbodiimide

DCM dichloromethane

DIC diisopropylcarbodiimide

EDC 1-ethyl-3-(-3-dimethylaminopropyl) carbodiimide hydrochloride

HMMM hexamethoxymethylmelamine

In(MA)₃ indium myristate

QD Quantum Dots

sulfo-NHS sulfo derivative of N-hydroxysuccinimide

SMCC succinimidyl 4-(N-maleimidomethyl)cyclohexane-1-carboxylate

(TMS)₃P tris(trimethylsilyl) phosphine

The terms such as “a,” “an,” and “the” are not intended to refer to a singular entity unless explicitly so defined, but include the general class of which a specific example may be used for illustration. The use of the terms “a” or “an” when used in conjunction with “comprising” in the claims and/or the specification may mean “one” but may also be consistent with “one or more,” “at least one,” and/or “one or more than one.”

The use of the term “or” in the claims is used to mean “and/or” unless explicitly indicated to refer to alternatives as mutually exclusive. Thus, unless otherwise stated, the term “or” in a group of alternatives means “any one or combination of” the members of the group. Further, unless explicitly indicated to refer to alternatives as mutually exclusive, the phrase “A, B, and/or C” means embodiments having element A alone, element B alone, element C alone, or any combination of A, B, and C taken together.

Similarly, for the avoidance of doubt and unless otherwise explicitly indicated to refer to alternatives as mutually exclusive, the phrase “at least one of” when combined with a list of items, means a single item from the list or any combination of items in the list. For example, and unless otherwise defined, the phrase “at least one of A, B and C,” means “at least one from the group A, B, C, or any combination of A, B and C.” Thus, unless otherwise defined, the phrase requires one or more, and not necessarily not all, of the listed items.

The terms “comprising” (and any form thereof such as “comprise” and “comprises”), “having” (and any form thereof such as “have” and “has”), “including” (and any form thereof such as “includes” and “include”) or “containing” (and any form thereof such as “contains” and “contain”) are inclusive or open-ended and do not exclude additional, unrecited elements or method steps.

The term “effective” as used in the specification and claims, means adequate to provide or accomplish a desired, expected, or intended result.

The terms “about” or “approximately” are defined as being close to as understood by one of ordinary skill in the art, and in one non-limiting embodiment the terms are defined to be within 10%, within, 5%, within 1%, and in certain aspects within 0.5%.

QDs are fluorescent semiconductor nanoparticles with unique optical properties. QD represent a particular very small size form of semiconductor material in which the size and shape of the particle results in quantum mechanical effects upon light excitation. Generally, larger QDs such as having a radius of 5-6 nm will emit longer wavelengths in orange or red emission colors and smaller QDs such as having a radius of 2-3 nm emit shorter wavelengths in blue and green colors, although the specific colors and sizes depend on the composition of the QD. QDs shine around 20 times brighter and are many times more photo-stable than any of the conventional fluorescent dyes (like indocyanine green (ICG)). Importantly, QD residence times are longer due to their chemical nature and nano-size. QDs can absorb and emit much stronger light intensities. In certain embodiments, the QD can be equipped with more than one binding tag, forming bi- or tri-specific nano-devices. The unique properties of QDs enable several medical applications that serve unmet needs.

In embodiments presented herein, the QDs are functionalized to present a hydrophilic outer layer or corona that permits use of the QDs in the aqueous environment, such as, for example, in vivo and in vitro applications in living cells. Such QDs are termed water soluble QDs.

In one embodiment the QDs may be surface equipped with a conjugation capable function (for example, COOH, OH, NH₂, SH, azide, alkyne). In one exemplified embodiment, the water soluble non-toxic QD is or becomes carboxyl functionalized. For example, the COOH-QD may be linked to the amine terminus of a targeting antibody using a carbodiimide linking technology employing water-soluble 1-ethyl-3-(-3-dimethylaminopropyl) carbodiimide hydrochloride (EDC). The carboxyl functionalized QD is mixed with EDC to form an active O-acylisourea intermediate that is then displaced by nucleophilic attack from primary amino groups on the monoclonal antibody in the reaction mixture. If desired, a sulfo derivative of N-hydroxysuccinimide (sulfo-NHS) is added during the reaction with the primary amine bearing antibody. With the sulfo-NHS addition, the EDC couples NHS to carboxyls, forming an NHS ester that is more stable than the O-acylisourea intermediate while allowing for efficient conjugation to primary amines at physiologic pH. In either event, the result is a covalent bond between the QD and the antibody. Other chemistries like Suzuki-Miyaura cross-coupling, (succinimidyl 4-(N-maleimidomethyl)cyclohexane-1-carboxylate) (SMCC), or aldehyde based reactions may alternatively be used.

Methods of synthesizing core and core-shell nanoparticles are disclosed, for example, in co-owned U.S. Pat. Nos. 7,867,556, 7,867,557, 7,803,423, 7,588,828, and 6,379,635. The contents of each of the forgoing patents are hereby incorporated by reference, in their entirety. U.S. Pat. Nos. 9,115,097, 8,062,703, 7,985,446, 7,803,423, and 7,588,828, and U.S. Publication Nos. 2010/0283005, 2014/0264196, 2014/0277297 and 2014/0370690, the entire contents of each of which are hereby incorporated by reference, describe methods of producing large volumes of high quality monodisperse QDs.

In one embodiment, a core/shell particle is utilized having a central region or “core” of at least one semiconductor composition buried in or coated by one or more outer layers or “shell” of distinctly different semiconductor compositions. As an example, the core may be comprised of an alloy of In, P, Zn and S such as is formed by the description of Example 1 involving molecular seeding of indium-based QDs over a ZnS molecular cluster followed by formation of a shell of ZnS.

In still other embodiments, the water soluble QD nanoparticle employed comprises an alloyed semiconductor material having a bandgap value or energy (E_{g}) that increases outwardly by graded alloying in lieu of production of a core/shell QD. The band gap energy (E_{g}), is the minimum energy required to excite an electron from the ground state valence energy band into the vacant conduction energy band.

The graded alloy QD composition is considered “graded” in elemental composition from at or near the center of the particle to the outermost surface of the QD rather than formed as a discrete core overlaid by a discrete shell layer. An example would be an In₁₋ₓP_{1-y}ZₙₓS_{y}, graded alloy QD wherein the x and y increase gradually from 0 to 1 from the center of the QD to the surface. In such example, the band gap of the QD would gradually change from that of pure InP towards the center to that of a larger band gap value of pure ZnS at the surface. Although the band gap of a nanoparticle is dependent on particle size, the bulk band gap of ZnS is wider than that of InP such that the band gap of the graded alloy would gradually increase from an inner aspect of the QD to the surface.

A one-pot synthesis process may be employed as a modification of the molecular seeding process described in Example 1 herein. This may be achieved by gradually decreasing the amounts of indium myristate and (TMS)₃P added to the reaction solution to maintain particle growth, while adding increasing amounts of zinc and sulfur precursors during a process such as is described for generation of the “core” particle of Example 1. Thus, in one example a dibutyl ester and a saturated fatty acid are placed into a reaction flask and degassed with heating. Nitrogen is introduced and the temperature is increased. A molecular cluster, such as for example a ZnS molecular cluster [Et₃NH]₄ [Zn₁₀S₄(SPh)₁₆], is added with stirring. The temperature is increased as graded alloy precursor solutions are added according to a ramping protocol that involves addition of gradually decreasing concentrations of a first semiconductor material and gradually increasing concentrations of a second semiconductor material. For example, the ramping protocol may begin with additions of indium myristate (In(MA)₃) and tris(trimethylsilyl) phosphine (TMS)₃P dissolved in a dicarboxylic acid ester (such as for example di-n-butylsebacate ester) wherein the amounts of added In(MA)₃ and (TMS)₃P gradually decrease over time to be replaced with gradually increasing concentration of sulfur and zinc compounds such as (TMS)₂S and zinc acetate. As the added amounts of In(MA)₃ and (TMS)₃P decrease, gradually increasing amounts of (TMS)₂S dissolved in a saturated fatty acid (such as for example myristic or oleic acid) and a dicarboxylic acid ester (such as di-n-butyl sebacate ester) are added together with the zinc acetate. The following reactions will result in the increasing generation of ZnS compounds. As the additions continue, QD particles of a desired size with an emission maximum gradually increasing in wavelength are formed wherein the concentrations of InP and ZnS are graded with the highest concentrations of InP towards a center of the QD particle and the highest concentrations of ZnS on an outer layer of the QD particle. Further additions to the reaction are stopped when the desired emission maximum is obtained and the resultant graded alloy particles are left to anneal followed by isolation of the particles by precipitation and washing.

A nanoparticle's compatibility with a medium as well as the nanoparticle's susceptibility to agglomeration, photo-oxidation and/or quenching, is mediated largely by the surface composition of the nanoparticle. The coordination about the final inorganic surface atoms in any core, core-shell or core-multi shell nanoparticle may be incomplete, with highly reactive “dangling bonds” on the surface, which can lead to particle agglomeration. This problem is overcome by passivating (capping) the “bare” surface atoms with protecting organic groups, referred to herein as capping ligands or a capping agent. The capping or passivating of particles prevents particle agglomeration from occurring but also protects the particle from its surrounding chemical environment and provides electronic stabilization (passivation) to the particles, in the case of core material. The capping ligands may be but are not limited to a Lewis base bound to surface metal atoms of the outermost inorganic layer of the particle. The nature of the capping ligand largely determines the compatibility of the nanoparticle with a particular medium. Capping ligand may be selected depending on desired characteristics. Types of capping ligands that may be employed include, but are not restricted to, thiol groups, carboxyl, amine, phosphine, phosphine oxide, phosphonic acid, phosphinic acid, imidazole, OH, thio ether, and calixarene groups. With the exception of calixarenes, all of these capping ligands have head groups that can form anchoring centers for the capping ligands on the surface of the particle. The body of the capping ligand can be a linear chain, cyclic, or aromatic. The capping ligand itself can be large, small, oligomeric or polydentate. The nature of the body of the ligand and the protruding side that is not bound onto the particle, together determine if the ligand is hydrophilic, hydrophobic, amphiphilic, negative, positive or zwitterionic.

In many QD materials, the capping ligands are hydrophobic (for example, alkyl thiols, fatty acids, alkyl phosphines, alkyl phosphine oxides, and the like). Thus, the nanoparticles are typically dispersed in hydrophobic solvents, such as toluene, following synthesis and isolation of the nanoparticles. Such capped nanoparticles are typically not dispersible in more polar media. If surface modification of the QD is desired, the most widely used procedure is known as ligand exchange. Lipophilic ligand molecules that coordinate to the surface of the nanoparticle during core synthesis and/or shelling procedures may subsequently be exchanged with a polar/charged ligand compound. An alternative surface modification strategy intercalates polar/charged molecules or polymer molecules with the ligand molecules that are already coordinated to the surface of the nanoparticle. However, while certain ligand exchange and intercalation procedures render the nanoparticle more compatible with aqueous media, they may result in materials of lower quantum yield (QY) and/or substantially larger size than the corresponding unmodified nanoparticle.

For in vivo and in vitro purposes, QDs with low toxicity profiles are desirable if not required. Thus, for some purposes, the QD is preferably substantially free of toxic heavy metals such as cadmium, lead and arsenic (e.g., contains less than 5 wt. %, such as less than 4 wt. %, less than 3 wt. %, less than 2 wt. %, less than 1 wt. %, less than 0.5 wt. %, less than 0.1 wt. %, less than 0.05 wt. %, or less than 0.01 wt. % of heavy metals such as cadmium, lead and arsenic) or is free of heavy metals such as cadmium, lead and arsenic. In one embodiment, reduced toxicity QDs that lack heavy metals such as cadmium, lead and arsenic are provided.

The unique properties of QDs enable several potential medical applications including unmet in vitro and in vivo diagnostics in living cells. One of the major concerns regarding the medical applications of QDs has been that the majority of research has focused on QDs containing toxic heavy metals such as cadmium, lead or arsenic. The biologically compatible and water-soluble heavy metal-free QDs described herein can safely be used in medical applications both in vitro and in vivo. In certain embodiments, in vivo compatible water dispersible cadmium-free QDs are provided that have a hydrodynamic size of 10-20 nm (within the range of the dimensional size of a full IgG2 antibody). In one embodiment, the in vivo compatible water dispersible cadmium-free QDs are produced in accordance with the procedures set out in Examples 1 and 2 herein. In certain embodiments, the in vivo compatible water dispersible cadmium-free QDs are carboxyl functionalized and further derivatized with a ligand binding moiety.

Examples of cadmium, lead and arsenic free nanoparticles include nanoparticles comprising semiconductor materials, e.g., ZnS, ZnSe, ZnTe, InP, InSb, AlP, AlS, AlSb, GaN, GaP, GaSb, PbS, PbSe, AgInS₂, CuInS₂, Si, Ge, and alloys and doped derivatives thereof, particularly, nanoparticles comprising cores of one of these materials and one or more shells of another of these materials.

In certain embodiments, non-toxic QD nanoparticles are surface modified to enable them to be water soluble and to have surface moieties that allow derivatization by exposing them to a ligand interactive agent to effect the association of the ligand interactive agent and the surface of the QD. The ligand interactive agent can comprise a chain portion and a functional group having a specific affinity for, or reactivity with, a linking/crosslinking agent, as described below. The chain portion may be, for example, an alkane chain. Examples of functional groups include nucleophiles such as thio groups, hydroxyl groups, carboxamide groups, ester groups, and a carboxyl groups. The ligand interactive agent may, or may not, also comprise a moiety having an affinity for the surface of a QD. Examples of such moieties include thiols, amines, carboxylic groups, and phosphines. If the ligand interactive group does not comprise such a moiety, the ligand interactive group can associate with the surface of the nanoparticle by intercalating with capping ligands. Examples of ligand interactive agents include C₈₋₂₀ fatty acids and esters thereof, such as for example isopropyl myristate.

It should be noted that the ligand interactive agent may be associated with a QD nanoparticle simply as a result of the processes used for the synthesis of the nanoparticle, obviating the need to expose nanoparticle to additional amounts of ligand interactive agents. In such case, there may be no need to associate further ligand interactive agents with the nanoparticle. Alternatively, or in addition, QD nanoparticle may be exposed to ligand interactive agent after the nanoparticle is synthesized and isolated. For example, the nanoparticle may be incubated in a solution containing the ligand interactive agent for a period of time. Such incubation, or a portion of the incubation period, may be at an elevated temperature to facilitate association of the ligand interactive agent with the surface of the nanoparticle. Following association of the ligand interactive agent with the surface of nanoparticle, the QD nanoparticle is exposed to a linking/crosslinking agent and a surface modifying ligand. The linking/crosslinking agent includes functional groups having specific affinity for groups of the ligand interactive agent and with the surface modifying ligand. The ligand interactive agent-nanoparticle association complex can be exposed to a linking/crosslinking agent and surface modifying ligand sequentially. For example, the nanoparticle might be exposed to the linking/crosslinking agent for a period of time to effect crosslinking, and then subsequently exposed to the surface modifying ligand to incorporate it into the ligand shell of the nanoparticle. Alternatively, the nanoparticle may be exposed to a mixture of the linking/crosslinking agent and the surface-modifying ligand thus effecting crosslinking and incorporating surface modifying ligand in a single step.

In one embodiment, QD precursors are provided in the presence of a molecular cluster compound under conditions whereby the integrity of the molecular cluster is maintained and acts as a well-defined prefabricated seed or template to provide nucleation centers that react with the chemical precursors to produce high quality nanoparticles on a sufficiently large scale for industrial application.

Suitable types of QDs useful in the present invention include, but are not limited to, core materials comprising the following types (including any combination or alloys or doped derivatives thereof):

IIA-VIB (2-16) material, incorporating a first element from group 2 of the periodic table and a second element from group 16 of the periodic table and also including ternary and quaternary materials and doped materials. Suitable nanoparticle materials include, but are not limited to: MgS, MgSe, MgTe, CaS, CaSe, CaTe, SrS, SrSe, SrTe, BaS, BaSe, BaTe.

II-V material incorporating a first element from group 12 of the periodic table and a second element from group 15 of the periodic table and also including ternary and quaternary materials and doped materials. Suitable nanoparticle materials include, but are not limited to: Zn₃P₂, Zn₃As₂, Cd₃P₂, Cd₃As₂, Cd₃N₂, Zn₃N₂.

II-VI material incorporating a first element from group 12 of the periodic table and a second element from group 16 of the periodic table and also including ternary and quaternary materials and doped materials. Suitable nanoparticle materials include, but are not limited to: CdSe, CdTe, ZnS, ZnSe, ZnTe, ZnO, HgS, HgSe, HgTe, CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, CdZnSeS, CdZnSeTe, CdHgSeS, CdHgSeTe, CdHgSTe, HgZnSeS, and HgZnSeTe.

III-V material incorporating a first element from group 13 of the periodic table and a second element from group 15 of the periodic table and also including ternary and quaternary materials and doped materials. Suitable nanoparticle materials include, but are not limited to: BP, AlP, AlSb; GaN, GaP, GaSb; InN, InP, InSb, AlN, and BN.

III-IV material incorporating a first element from group 13 of the periodic table and a second element from group 14 of the periodic table and also including ternary and quaternary materials and doped materials. Suitable nanoparticle materials include, but are not limited to: B₄C, Al₄C₃, Ga₄C, Si, SiC.

III-VI material incorporating a first element from group 13 of the periodic table and a second element from group 16 of the periodic table and also including ternary and quaternary materials. Suitable nanoparticle materials include, but are not limited to: Al₂S₃, Al₂Se₃, Al₂Te₃, Ga₂S₃, Ga₂Se₃, GeTe; In₂₅₃, In₂Se₃, Ga₂Te₃, In₂Te₃, InTe.

IV-VI material incorporating a first element from group 14 of the periodic table and a second element from group 16 of the periodic table, and also including ternary and quaternary materials and doped materials. Suitable nanoparticle materials include, but are not limited to: PbS, PbSe, PbTe, Sb₂Te₃, SnS, SnSe, SnTe.

Suitable nanoparticle material can incorporate a first element from any group in the transition metal of the periodic table and a second element from group 16 of the periodic table and also including ternary and quaternary materials and doped materials. For example, a material incorporates a first element from group 11 of the periodic table, a second element from group 13 of the periodic table and a third element from group 16 of the periodic table, and including quaternary, higher order and doped materials. Suitable nanoparticle materials include, but are not limited to: CuInS₂, CuInSe₂, CuGaS₂, CuGaSe₂, AgInS₂, AgInSe₂, NiS, CrS and AgS.

In one embodiment, the QDs useful in the present invention include, but are not limited to, core materials comprising AgS.

In one embodiment of any of the QDs described herein, the nanoparticle comprises a II-IV material, a III-V material, a material, or any alloy or doped derivative thereof.

In one embodiment, the nanoparticle material comprises a II-IV material, a III-V material, and any alloy or doped derivative thereof.

In one embodiment of any of the QDs described herein, the nanoparticle comprises a III-V material, or any alloy or doped derivative thereof.

The term doped nanoparticle for the purposes of specifications and claims refers to nanoparticles of the above and a dopant comprising one or more main group or rare earth elements, this most often is a transition metal or rare earth element, such as but not limited to zinc sulfide with manganese, such as ZnS nanoparticles doped with Mn⁺.

In one embodiment, the QDs is substantially free of heavy metals such as cadmium (e.g., contains less than 5 wt. %, such as less than 4 wt. %, less than 3 wt. %, less than 2 wt. %, less than 1 wt. %, less than 0.5 wt. %, less than 0.1 wt. %, less than 0.05 wt. %, or less than 0.01 wt. % of heavy metals such as cadmium) or is free of heavy metals such as cadmium.

For in vivo applications, heavy metal-free semi-conductor indium-based nanoparticles or nanoparticles containing indium and/or phosphorus are preferred.

In an embodiment, any of the QDs described herein include a first layer including a first semiconductor material provided on the nanoparticle core. A second layer including a second semiconductor material may be provided on the first layer.

### Synthesis

The following synthesis steps may be used for conjugation. Linkers may be used to form an amide group between the carboxyl functions on the nanoparticles and the amine end groups on the cancer-specific binding ligand. Known linkers, such as a thiol anchoring groups directly on the inorganic surface of the QDs can be used. Standard coupling conditions can be employed and will be known to a person of ordinary skill in the art. For example, suitable coupling agents include, but are not limited to, carbodiimides, such as dicyclohexylcarbodiimide (DCC), diisopropylcarbodiimide (DIC), and 1-(3-dimethylaminopropyl)-3-ethylcarbodiimide hydrochloride (EDC). In one embodiment, the coupling agent is EDC.

In an example, the QDs bearing a carboxyl end group and a polymerizable ligand may be mixed in a solvent. A coupling agent, such as EDC, may be added to the mixture. The reaction mixture may be incubated. The crude polymerizable ligand nanoparticle conjugate may be subject to purification and/or isolated.

Standard solid state purification methods may be used. Several cycles of filtering and washing with a suitable solvent may be necessary to remove excess unreacted functionalized ligand and/or coupling agents.

In another aspect, one embodiment provides a process for preparing a ligand nanoparticle conjugate for according to any of the embodiments described herein. In one embodiment, the process comprises: i) coupling a QDs with a polymerizable ligand to give a ligand-nanoparticle conjugate, wherein the nanoparticle comprises a core semiconductor material, and an outer layer, wherein the outer layer comprises a carboxyl group. In one embodiment, coupling step i) comprises (a) reacting a carboxyl group in the outer layer with a carbodiimide linker to activate the carboxyl group, and b) reacting the activated carboxyl group with a polymerizable ligand.

In an additional embodiment, the process further comprises: ii) purifying the ligand nanoparticle conjugate. In an additional embodiment, the process further comprises: iii) isolating the ligand nanoparticle conjugate. In one embodiment, the process comprises steps i), ii) and iii).

In an additional embodiment, the process further comprises: ii) purifying the QDs. In an additional embodiment, the process further comprises: iii) isolating the specific binding nanoparticle conjugate. In one embodiment, the process comprises steps i), ii) and iii).

### Examples

Standard conjugation chemistry may be used for conjugation. For example, a method preparing a nanoparticle polymerizable ligand conjugate may include the steps of providing a nanoparticle, providing a coupling agent, providing a polymerizable ligand, such as, for example, 2-aminoethyl methacrylate hydrochloride, and incubating the mixture to form a nanoparticle polymerizable ligand conjugate. The mixture may then be purified and isolated to obtain a nanoparticle polymerizable ligand conjugate.

The incubation conditions may be chosen to allow for formation of either an amide or an ester. It should be understood that other bonds may be formed (e.g., both covalent and non-covalent). The polymerizable ligand can be conjugated with the nanoparticle either covalently, physically, ion pairing, or van der Waals interactions. The bond may be formed by an amide, ester, thioester, or thiol anchoring group directly on the inorganic surface of the QD, or on the organic corona layer that is used to render the nanoparticles water soluble and biocompatible.

Standard incubation conditions for coupling can be employed. For example, the coupling conditions may be a solution in the range of 0.5 to 4 hours. The temperature range of the coupling conditions may be in the range of 0° C. to 200° C. The coupling conditions may be constant or varied during the reaction. For example, the reaction conditions may be 130° C. for one hour then raised to 140° C. for three hours.

In an example, the QDs bearing a carboxyl end group and polymerizable ligand may be mixed in a solvent. A coupling agent, such as EDC, may be added to the mixture. The reaction mixture may be incubated. A crude polymerizable ligand—QD nanoparticle conjugate may be subjected to purification to obtain the conjugated QD nanoparticle conjugate.

Standard solid state purification method may be used. Several cycles of filtering and washing with a suitable solvent may be necessary to remove excess unreacted polymerizable ligand and EDC.

The nanoparticle polymerizable ligand conjugate can subsequently be introduced into a mammal or tissue for real-time imaging and treatment of affected tissues. The administration of the polymerizable ligand conjugate can be enteral or parenteral. For example, the polymerizable ligand conjugate can be administered subcutaneously, intravenously, intramuscular, topically, and orally. Examples include bolus injections or IV infusions.

### Preparation of Functionalized Quantum Dot (QD) Conjugates

Carboxy functionalized QDs are linked to 2-aminoethyl methacrylate hydrochloride using standard EDC chemistry. The resulting dots have pendant methacrylate groups that are delivered to the targeted tissue and polymerized by the excitations of the QDs with an energy source.

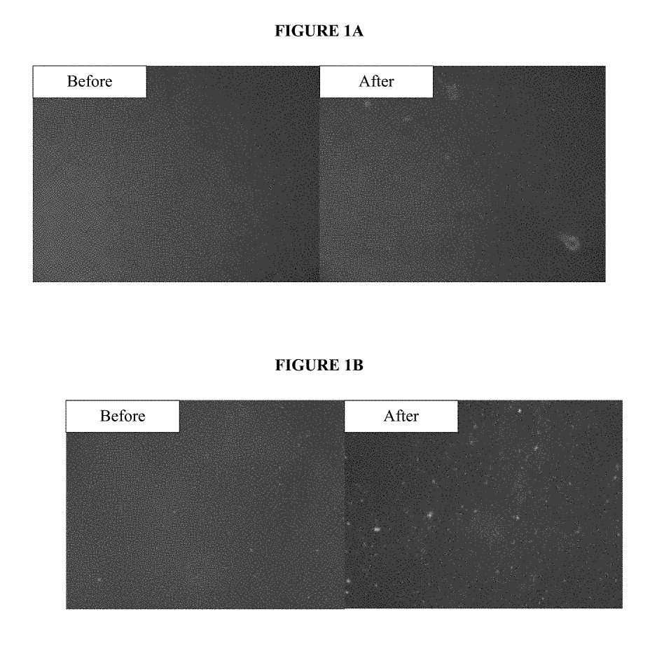

Carboxy functionalized red QDs were linked to methacryloyl-L-lysine using standard EDC chemistry. The resulting QDs have pendant methacrylate groups that are polymerizable by UV/visible excitation at 300-500 nm. Fluorescence microscopy imaging at 1000× magnification showed that when exposed to 320 nm UV, the nanoparticles aggregated, unlike the ones that were not irradiated. See FIG. 1A. The QDs can be delivered to the targeted tissue and polymerized by the excitations of the QDs with an energy source

In one example, carboxy functionalized QDs were surface loaded with 4-methacryloxy-2-hydroxybenzophenone (Formula I) using hydrophobic interaction forces as follows. To an amount of 100 mg water soluble dots (Vivodots™ 630 nanoparticles (Nanoco Technologies Limited, Manchester, UK)) dispersed in 1 mL H₂O, a 1004, solution of 4-methacryloxy-2-hydroxybenzophenone dissolved in DMSO at 100 mg/mL was added with vigorous mixing.

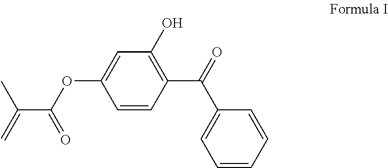 [SMILES: C=C(C)C(=O)Oc1ccc(C(=O)c2ccccc2)c(O)c1]

A clear solution was formed and to which 1 mL of phosphate buffered saline (PBS, pH7.2) was immediately added. The solution remained clear despite the fact that 4-methacryloxy-2-hydroxybenzophenone is insoluble in water. This is an indication that the monomer 4-methacryloxy-2-hydroxybenzophenone was able to form hydrophobic interactions on the surface of the nanoparticles and became dispersed with them. The clear solution was then sterilized using 0.22 um syringe filter.

A small drop of the polymerizable QD preparation was mounted on a microscope slide, covered with a glass coverslip, and then irradiated for 5 minutes using a 6 Watt handheld UV lamp (UVP, LLC) at 365 nm wavelength. A control slide was prepared in the same manner but was not irradiated. The slides were then examined using a fluorescence microscope. As shown in FIG. 1B, the irradiated sample showed significant aggregation.

### Ablation of Unwanted Cells

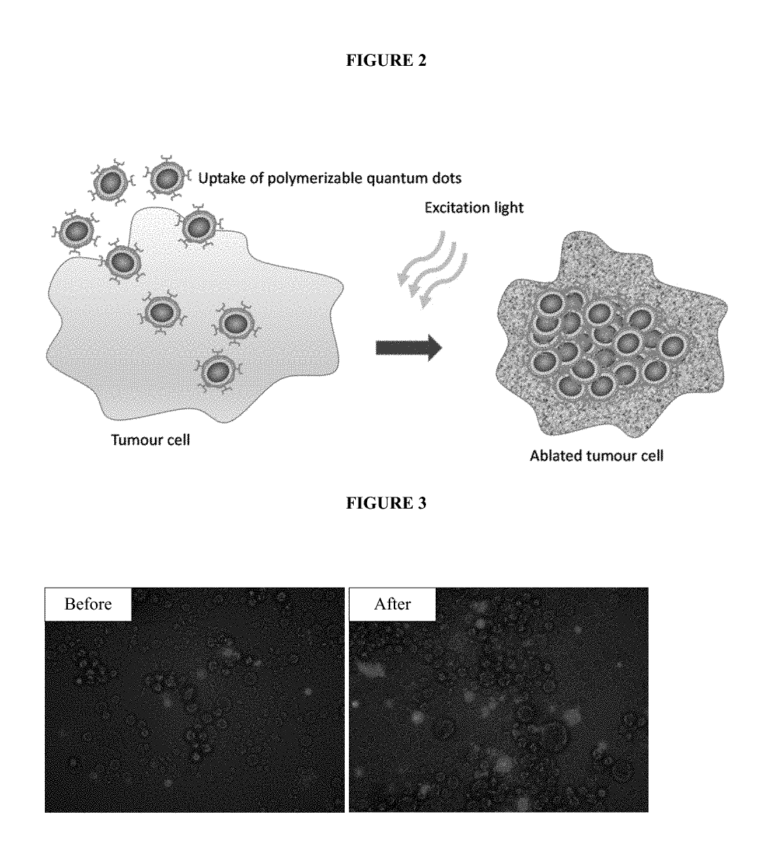

The QD polymerizable ligand conjugates are taken up by tumor cells. Excitation of the QD conjugates by an external light source triggers polymerization of the polymerizable ligand causing dot-dot crosslinking that leads to intra-tissue aggregation and tissue necrosis or death. See FIG. 2.

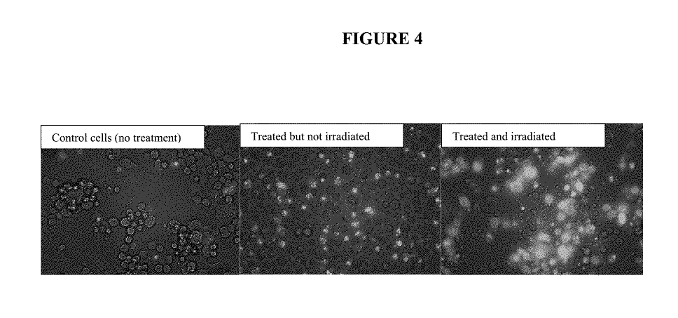

In one example, SKBR3 human breast cells were cultivated in McCoy's medium supplemented with 10% fetal bovine serum and a sample of polymerizable QDs (poly Vivodots™ 630 nanoparticles) was added at 0.5 mg/mL in PBS buffer. After 24 hrs incubation to allow cellular uptake, the cells were irradiated using the light source of a Zeiss microscope (Zeiss Axiovert 200 m) using the Texas red filter and a 20× objective lens. Only irradiated cells showed significant cell damage as confirmed using DAPI staining as shown in FIGS. 3 and 4.

Accordingly, the polymerizable QDs are capable of being applied to specified unwanted cells within mammalian tissue, such as, for example, tumors. The polyermizable QDs are taken up by the cells targeted, and, upon irradiation with a light source (either from the QDs themselves or an external source), cause polymerization and cell death. Other example of unwanted cells include soft, solid, malignant, and benign tumors. Other examples include varicose veins, telangiectasia, and spider nevus (spider veins).

The polymerization also aids in the imaging of the unwanted cells within mammalian tissue.

### Use of the Quantum Dot Conjugates for Cosmetic Tattooing

The QD polymerizable ligand conjugates can also be used for cosmetic tattooing. In this embodiment, the QD polymerizable ligand conjugates are delivered to the desired tissue (e.g., dermis and epidermis). In this embodiment, the polymerizable ligand encapsulates a pigmented ink. Once delivered, the polymerizable ligands can disassociate from the nanoparticle, and subsequently be excited by irradiating light from the nanoparticle, resulting in polymerization. The polymerization of the ligands will result in a structure that is visible through the skin.

In addition, both blacklight and glow in the dark inks have been used for tattooing. Glow in the dark ink absorbs and retains light, and then glows in darkened conditions by process of phosphorescence. Blacklight ink does not glow in the dark, but reacts to non-visible UV light, producing a visible glow by fluorescence. A typical glow ink comprises polymethylmethacrylate (97.5%) and microspheres of fluorescent dye (2.5%).

The QD polymerizable ligand conjugates are injected into the skin. Once delivered, the ligands can disassociate from the nanoparticle, and subsequently be excited by irradiating light from the nanoparticle, resulting in polymerization. In the case of blacklight and glow in the dark inks, further excitation by UV light, for example, can be administered to cause fluorescence.

It should be understood that the cosmetic tattooing can be reversible by disruption of the bonds between the ligands. This can be performed by the application of appropriate energy to safely disrupt the bonds in a mammal.

These and other advantages of the present invention will be apparent to those skilled in the art from the foregoing specification. Accordingly, it is to be recognized by those skilled in the art that changes or modifications may be made to the above-described embodiments without departing from the broad inventive concepts of the invention. It is to be understood that this invention is not limited to the particular embodiments described herein, but is intended to include all changes and modifications that are within the scope and spirit of the invention.

What is claimed is:

1. A quantum dot nanoparticle conjugate, wherein the nanoparticle is linked to a polymerizable ligand.

2. The quantum dot nanoparticle conjugate of claim 1, wherein the quantum dot nanoparticle comprises:
a core semiconductor material, and
an outer layer,
wherein the outer layer comprises a functionalization organic coating linked to a polymerizable ligand.

3. The quantum dot nanoparticle conjugate of claim 1, wherein the polymerizable ligand is selected from the group consisting of acrylates, methacrylates, diacetylene, cyanoacrylates, azide/alkyne pairs, and any combination thereof.

4. The quantum dot nanoparticle conjugate of claim 1, wherein the polymerizable ligand comprises acrylates and methacrylates.

5. The quantum dot nanoparticle conjugate of claim 1, wherein the polymerizable ligand is a cyanoacrylate.

6. The quantum dot nanoparticle conjugate of claim 1, wherein the polymerizable ligand is a diacetylene.

7. The quantum dot nanoparticle conjugate of claim 1 wherein the polymerizable ligand is polymerized by a chemical or physical action.

8. The quantum dot nanoparticle conjugate of claim 1, wherein each quantum dot nanoparticle comprises a II-VI material, a III-V material, a I-III-VI material, or any alloy or doped derivative thereof.

9. The quantum dot nanoparticle conjugate of claim 1, wherein the quantum dot nanoparticle further comprises a cellular uptake enhancer, a tissue penetration enhancer, or a combination thereof.

10. A method of inducing cell death comprising
i) contacting a quantum dot nanoparticle conjugate according to claim 1 with a cell;
ii) triggering polymerization of the polymerizable ligand using a chemical or physical action.

11. The method of claim 10, wherein the cell death is induced upon polymerization and affected tissue is visualized.

12. A method of treating and visualizing a tumor comprising
i) contacting a quantum dot nanoparticle conjugate according to claim 1 with a tumor;
ii) triggering polymerization of the polymerizable ligand using a chemical or physical action.

13. A method of ablating unwanted tissue comprising
i) contacting a quantum dot nanoparticle conjugate according to claim 1 with unwanted tissue;
ii) triggering polymerization of the polymerizable ligand using a chemical or physical action

14. The method of claim 13, wherein the unwanted tissue is selected from varicose veins, telangiectasia, and spider nevus.

15. The use of a quantum dot nanoparticle conjugate according to claim 1 for inducing cell death upon polymerization.

16. The use of a quantum dot nanoparticle conjugate according to claim 1 for inducing cell death upon polymerization and imaging affected tissues.

17. The use of a quantum dot nanoparticle conjugate according to claim 1 for the visualization and treatment of malignant and benign tumors.

18. The use of a quantum dot nanoparticle conjugate according to claim 1 for the visualization and treatment of soft and solid tumors.

19. The use of a quantum dot nanoparticle conjugate according to claim 1 for cosmetic tattooing.

20. The use of a quantum dot nanoparticle conjugate according to claim 1 for ablating unwanted tissue, varicose veins, telangiectasia, and spider nevus.
